(19) United States
(12) Patent Application Publication
(10) Pub. No.: US 2013/0183288 A1
(43) Pub. Date: Jul. 18, 2013

(54) NON-FUCOSYLATED ANTIBODIES

(71) Applicants: Mitchell Reff, San Diego, CA (US); Kandasamy Hariharan, San Diego, CA (US); MacLaren Ann, San Diego, CA (US); Sisk William, Boxborough, MA (US)

(72) Inventors: Mitchell Reff, San Diego, CA (US); Kandasamy Hariharan, San Diego, CA (US); MacLaren Ann, San Diego, CA (US); Sisk William, Boxborough, MA (US)

(73) Assignee: BIOGEN IDEC INC., Cambridge, MA (US)

(21) Appl. No.: 12/593,282

(22) PCT Filed: Mar. 28, 2008
(86) PCT No.: PCT/US08/58750

§ 371 Date: Apr. 29, 2010

Related U.S. Application Data
(60) Provisional application No. 60/908,643, filed on Mar. 28, 2007.

(51) Int. Cl. C07K 16/28; A61K 39/395; A61K 45/06

(52) U.S. Cl. CPC C07K 16/2851; A61K 45/06; C07K 16/2827; A61K 39/3955
USPC 424/133.1; 530/387.3; 435/328

(57) ABSTRACT

The present invention provides non-fucosylated therapeutic antibodies. More particularly, the invention provides non-fucosylated anti-CD20, anti-CD23 and anti-CD80 antibodies, which show enhanced antibody effector functions. Also provided are cells expressing the non-fucosylated antibodies and therapeutic methods using the non-fucosylated antibodies.

### RELATED APPLICATIONS

Priority is claimed from U.S. Provisional Application No. 60/908,643, filed Mar. 28, 2007, which is incorporated herein by reference in its entirety.

### FIELD OF THE INVENTION

The present invention provides non-fucosylated therapeutic antibodies. More particularly, the invention provides non-fucosylated anti-CD20, anti-CD23 and anti-CD80 antibodies, which show enhanced antibody effector functions. Also provided are cells expressing the non-fucosylated antibodies and therapeutic methods using the non-fucosylated antibodies.

### BACKGROUND OF THE INVENTION

Therapeutic antibodies are recognized as effective medicines which may improve overall survival time or limit disease progression in a variety of human malignancies and autoimmune conditions. The high affinity and specific binding of monoclonal antibodies has been the basis for the development of targeted immunotherapies, wherein a therapeutic molecule is conjugated to an antibody for delivery to cells expressing an antigen specifically bound by the antibody. Recombinant therapeutic antibodies have also been prepared, which incorporate effector functions of human antibody constant regions. Thus, therapeutic antibodies are effective in unlabeled or unconjugated form. For the treatment of malignancies, useful effector functions include an ability to induce antibody-dependent cellular cytotoxicity (ADCC) and an ability to activate complement. Effector functions such as inhibition of IgE are useful for treatment of autoimmune or allergy indications. Examples of therapeutic antibodies include rituximab (RITUXAN®), an anti-CD20 antibody, galiximab, an anti-CD80 antibody, and lumiliximab, an anti-CD23 antibody. RITUXAN® has demonstrated efficacy in patients with various lymphoid malignancies, including indolent and aggressive forms of B-cell non-Hodgkin's lymphoma (NHL) and B-cell chronic lymphocytic leukaemia (CLL). See, e.g., Plosker et al., Drugs, 2003, 63(8):803-43. Both galiximab and lumiliximab have shown success in clinical or preclinical trials for the treatment of B cell malignancies as well as autoimmune/allergic conditions. See e.g., Gottlieb et al., Clin. Immunol., 2004, 28-37; Czuczman et al., J. Clin. Oncol., 2005, 23(19):4390-4398; Rosenwasser et al., J. Allergy Clin. Immunol., 2003, 112(3):563-570; Poole et al., J. Allergy Clin. Immunol., 2005, 116(4):780-788; Pathan et al., ASCO Meeting, June 2007 (abstract).

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. (Jenkins et al., Nature Biotechnol., 1996, 14:975-981). Recent studies have also shown that glycosylation contributes to antibody effector functions. See e.g., Shinkawa et al., J. Biol. Chem., 2003, 278(5):3466-3473; Niwa et al., Clin. Cancer Res., 2004, 10(18 Pt 1):6248-6255; Natsume et al., J. Immunol. Methods, 2005, 306(1-2):93-103; Natsume et al., J. Biochem. (Tokyo), 2006, 140(3):359-368; Niwa et al., J. Immunol. Methods, 2005, 306(1-2):151-160; Suzuki et al., Clin. Cancer Res., 2007, 13(6):1875-1882; Iida et al., Clin. Cancer Res., 2006; 12(9):2879-2887.

There remains a continuing need to improve the efficacy of therapeutic antibodies so that durable remissions or complete recovery from disease can be achieved, particularly in patients with relapsed or refractory disease conditions. To meet this need, the present invention provides non-fucosylated anti-CD20, anti-CD80, and anti-CD23 antibodies demonstrating increased potency due to enhanced effector cell functions. Methods of preparing such antibodies are also provided.

### SUMMARY OF THE INVENTION

The present invention provides non-fucosylated anti-CD20, anti-CD23, and non-CD80 antibodies. Such antibodies include an Fc region and complex N-glycoside-linked sugar chains bound to the Fc region, wherein among the total complex N-glycoside-linked sugar chains bound to the Fc region, the ratio of a sugar chain in which fucose is not bound to N-acetylglucosamine in the reducing end in the sugar chain is at least 20%. The non-fucosylated antibodies have enhanced Fc receptor binding and enhanced effector functions as compared to control fucosylated antibodies. Also provided are cells expressing the non-fucosylated antibodies. Still further provided are methods of treating a neoplastic disorder or an autoimmune or allergic disorder by administering the disclosed non-fucosylated antibodies.

### BRIEF DESCRIPTION OF THE DRAWINGS

FIG. 1 depicts the general structure of human IgG1 antibody. The antibody is composed of two identical heavy chains and two identical light chains in covalent and non-covalent association to form three independent protein moieties connected through the hinge region. Human IgG1 has two N-linked oligosaccharides in the C_{H}2 domain in the Fc region. The general structure of human IgG1 N-linked oligosaccharides is characterized by a mannosyl core (Man3GlcNAc2-Asn) with or without a N-acetylglucosamine (GlcNAc) moiety with bound fucose (Fuc), and with or without additional chain moieties, such as galactose or sialic acid.

FIGS. 2A-2B depict the results of ⁵¹Cr antibody dependent cellular cytotoxicity assays as described in Example 2. The assays were used to assess the effect on CD23+ SKW B lymphoma cells contacted with fucosylated and non-fucosylated anti-CD23 antibodies in the presence of effector cells from Donor 128 (FIG. 2B) or Donor 127 (FIG. 2A). Percentage lysis is plotted as a function of antibody concentration (μg/ml). Non-fucosylated lumiliximab (squares) exhibited enhanced ADCC in comparison to control antibodies, fucosylated lumiliximab (triangles) and anti-CD4 antibody CE9.1 (inverted triangle).

FIGS. 3A-3B depict the results of ⁵¹Cr antibody dependent cellular cytotoxicity assays as described in Example 2. The assays were used to assess the effect on CD20+SKW B lymphoma cells contacted with fucosylated and non-fucosylated anti-CD20 antibodies in the presence of effector cells from Donor 128 (FIG. 3B) or Donor 127 (FIG. 3A). Percentage lysis is plotted as a function of antibody concentration (μg/ml). Non-fucosylated Rituximab (squares) exhibited enhanced ADCC in comparison to control antibodies, fucosylated Rituximab (triangles) and anti-CD4 antibody CE9.1 (inverted triangle).

FIGS. 4A-4B depict the results of ⁵¹Cr antibody dependent cellular cytotoxicity assays as described in Example 3. The assays were used to assess the effect on the CD80+ Non-Hodgkin's B-cell lymphoma (B-NHL) Raji cell line contacted with fucosylated and non-fucosylated anti-CD80 antibodies in the presence of effector cells. Percentage lysis is plotted as a function of antibody concentration (μg/ml). Non-fucosylated galiximab (circles) exhibited enhanced ADCC in comparison to control antibodies, fucosylated galiximab (squares) and anti-CD4 antibody CE9.1 (inverted triangle).

FIG. 5 depicts the results of ⁵¹Cr antibody dependent cellular cytotoxicity assays as described in Example 4. The assays were used to assess the effect on CD20+CD23+B-CLL cells isolated from CLL patients contacted with fucosylated and non-fucosylated anti-CD20 and anti-CD23 antibodies in the presence of effector cells. Percentage lysis is shown for a constant antibody concentration (1 nM) for various effector to target cell ratios. Non-fucosylated lumiliximab exhibited enhanced ADCC in comparison to fucosylated lumiliximab at an effector to target cell ratio of 50:1 (hatched bars) but not at effector to target cell ratios of 25:1 (black bars), 12.5:1 (grey bars) or in the absence of effector cells (white bars). Non-fucosylated rituximab exhibited some increased ADCC in comparison to fucosylated rituximab at an effector to target cell ratio of 50:1 (hatched bars) but not at effector to target cell ratios of 25:1 (black bars), 12.5:1 (grey bars) or in the absence of effector cells (white bars)

### DETAILED DESCRIPTION

The present invention provides non-fucosylated anti-CD20, anti-CD23 antibodies, and anti-CD80 antibodies, i.e., antibodies having the noted binding specificity and further comprising an Fc region to which oligosaccharides having reduced fucose moieties are bound. The non-fucosylated antibodies of the invention show enhanced antibody effector functions as compared to control fucosylated antibodies. The present invention also provides cells expressing non-fucosylated antibodies and methods for using the disclosed antibodies in therapeutic applications.

### I. Non-Fucosylated Antibodies

Naturally occurring antibodies comprise two identical light polypeptide chains, each having a molecular weight of approximately 23,000 Daltons, and two identical heavy chains, each having a molecular weight of 53,000-70,000 Daltons. The four chains associate to form three globular domains joined by a flexible hinge region. The variable domains of both the light (V_{L}) and heavy (V_{H}) chains determine antigen recognition and specificity. The constant domains of the light chain (C_{L}) and the heavy chain (C_{H}1, C_{H}2 or C_{H}3) confer biological properties such as Fc receptor binding, complement binding and the like. These effector functions are modulated by glycosylation of the Fc regions.

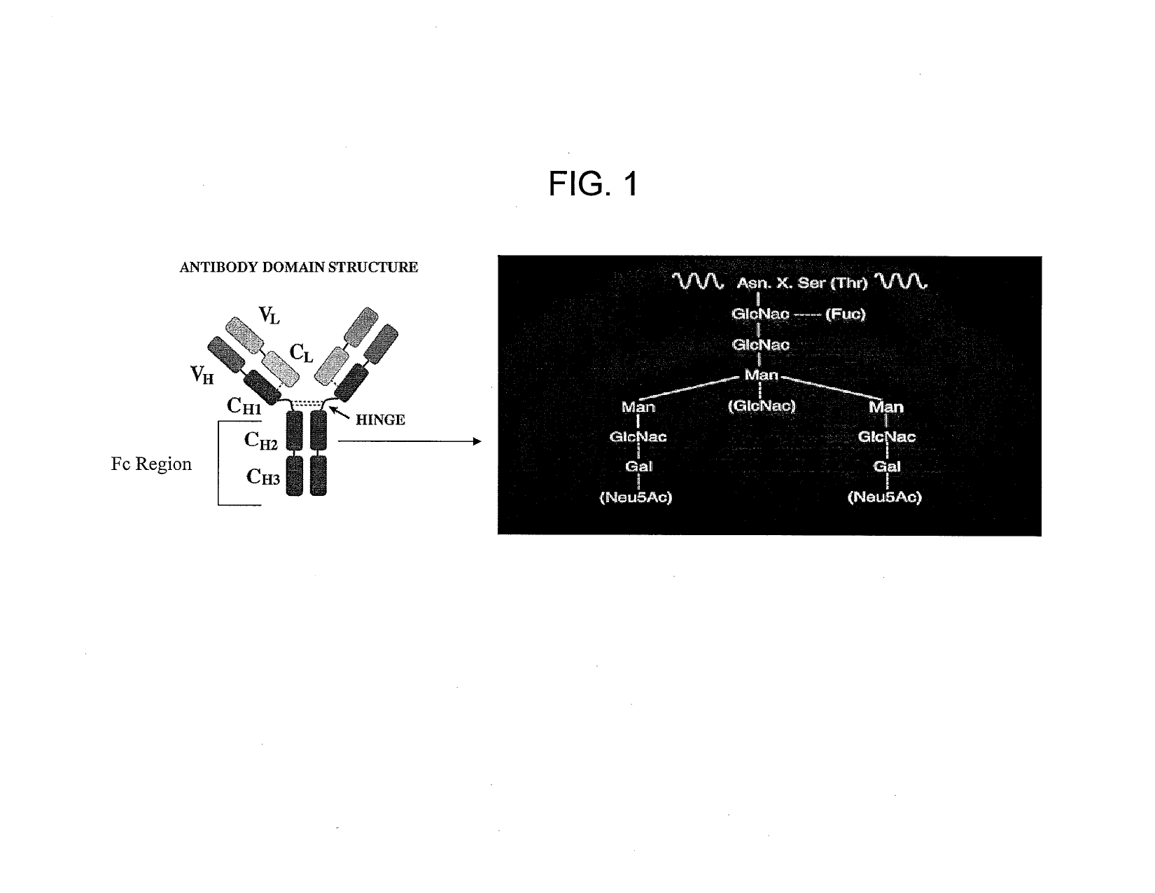

All antibodies contain carbohydrate structures at conserved positions in the heavy chain constant regions, with each isotype possessing a distinct array of N-linked carbohydrate structures, which variably affect protein assembly, secretion or functional activity. (Wright et al., Trends Biotech., 1997, 15:26-32. The oligosaccharide chains which are bound to the Fc regions of antibodies are characterized by a core structure comprised of GlcNAc and mannose, and optionally, a bisecting GlcNAc, as shown in FIG. 1. The structure of the attached N-linked carbohydrate varies considerably and can include high-mannose, multiply-branched as well as biantennary complex oligosaccharides. As a result of heterogeneous processing of the core oligosaccharide structures, antibodies typically exist as multiple glycoforms.

The oligosaccharide moieties present on antibodies include a reducing end and a non-reducing end. The reducing end of the oligosaccharide refers to the end of the core structure that is covalently attached to an amino acid residue within the Fc region of antibodies. The end opposite the reducing end is referred to as the non-reducing end. The oligosaccharides chains may be N-linked (aspargine-linked) oligosaccharide chains, wherein the oligosaccharide chain is covalently attached at the reducing end to an asparagine residue, such as Asn²⁹⁷, within the Fc region. The non-reducing end of the core structure of bound oligosaccharides may include a variety of chain variants. For example, oligosaccharide chains may be of the high mannose type, the complex type or the hybrid type. In the high mannose type, the core structure of the oligosaccharide chain is modified such that only additional mannose residues are bound to the non-reducing end of the core structure. The oligosaccharide chains may be of the complex type. That is, the non-reducing end of the core structure may have at least one parallel branch of galactose-N-acetylglucosamine (Gal-GlcNAc) and the Gal-GlcNAc parallel branch may further include a structure of sialic acid and/or a bisecting GlcNAc. Hybrid oligosaccharide chains have branches of both the high mannose type and the complex type structures at the non-reducing end side of the core structure. Other oligosaccharide chain variants are described in Takahashi, R. (ed), Biochemical Experimentation Method 23: Method for Studying Glycoprotein Sugar Chain, 1989, Gakujutsu Shuppan Center.

Non-fucosylated antibodies of the instant invention comprise one or more sugar chains in which fucose is not bound to N-acetylglucosamine in the reducing end in the complex X-glycoside-linked sugar chain, i.e., a sugar chain in which 1-position of the fucose is not bound to N-acetylglucosamine in the reducing end through α-bond in the complex N-glycoside-linked sugar chain. Examples include a complex N-glycoside-linked sugar chain in which 1-position of fucose is not bound to 6-position of N-acetylglucosamine through c-bond.

The ratio of a sugar chain in which fucose is not bound to N-acetylglucosamine in the reducing end in the sugar chain among the total complex N-glycoside-linked sugar chains binding to the Fc region contained in an antibody or antibody composition of the invention is at least about 20%, such as at least about 25%, or at least about 30%, at least about 40%, or at least about 50%, or at least about 60%, or at least about 70%, or at least about 80%, or at least about 90%, or at least about 95%, or at least about 99%. Antibodies and antibody compositions having the above-noted ratios of a sugar chain show enhanced antibody effector functions, as described further herein below.

The ratio of a sugar chain in which fucose is not bound to N-acetylglucosamine in the reducing end in the sugar chain contained in the composition which comprises an antibody molecule having complex N-glycoside-linked sugar chains in the Fc region can be determined by releasing the sugar chain from the antibody molecule using a known method such as hydrazinolysis, enzyme digestion (see e.g., Takahashi, P. (ed), Biochemical Experimentation Methods 23—Method for Studying Glycoprotein Sugar Chain, 1989, Japan Scientific Societies Press), carrying out fluorescence labeling or radioisotope labeling of the released sugar chain, and then separating the labeled sugar chain by chromatography. Alternatively, the released sugar chain can be detected using high-performance anion-exchange chromatographic method using pulsed amperometric detection (HPAEC-PAD), as described by Pohl, J. Liq. Chromatogr., 1983, 6, 1577.

Non-fucosylated anti-CD20, anti-CD23, and anti-CD80 antibodies as described herein include non-fucosylated versions of any such antibodies, e.g., antibodies produced in a cell deficient in fucose metabolism, as described below. Accordingly, non-fucosylated anti-CD20, anti-CD23, and anti-CD80 antibodies of the invention include polyclonal antibodies, monoclonal antibodies (mAbs), humanized antibodies, chimeric antibodies, PRIMATIZED® antibodies, which contain human constant regions and primate (cynomolgus macaque) variable regions, human monoclonal antibodies, single chain antibodies, and anti-idiotypic (anti-Id) antibodies. Such antibodies may have a structure of a naturally occurring antibody, multivalent forms thereof, or fragments thereof which include fragments comprising a CH2 domain and an ability to mediate antibody effector functions, as described further below.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 1975, 256:495-497; and U.S. Pat. No. 4,376,110), the human B cell hybridoma technique (Kosbor et al., Immunology Today, 1983, 4:72; Cote et al., Proc. Natl. Acad. Sci. USA, 1983, 80:2026-2030), and the EBV-hybridoma technique (Cole et al., in Monoclonal Antibodies And Cancer Therapy, 1995, Alan R. Liss, Inc., New York, pp. 77-96). Such antibodies may be of any immunoglobulin class including IgG, IgM, IgE, IgA, IgD and any subclass thereof. Hybridomas producing monoclonal antibodies may be cultivated in vitro or in vivo.

A chimeric antibody is a molecule in which different portions are derived from different animal species, such as those having a variable region derived from a murine mAb and a human immunoglobulin constant region. Chimeric antibodies may be produced (Morrison et al., Proc. Natl. Acad. Sci. USA, 1984, 81:6851-6855; Takeda et al., Nature, 1985, 314:452-454) by splicing the genes from a mouse antibody molecule of appropriate antigen specificity together with genes from a human antibody molecule of appropriate biological activity.

A humanized antibody is a type of chimeric antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. These general approaches may be combined with standard mutagenesis and synthesis techniques to produce an antibody of any desired sequence.

The antibodies of the present invention may also be human monoclonal antibodies, for example those produced by immortalized human cells, by SCID-hu mice or other non-human animals capable of producing human antibodies. Human antibodies may also be isolated from antibody phage libraries, for example, as described by Marks et al., J. Mol. Biol., 1991, 222:581-597. Chain shuffling and recombination techniques may be used to produce phage libraries having increased antibody diversity, e.g., libraries including antibodies with increased binding affinity. See Marks et al., Biotechnology, 1992, 10:779-783 and Waterhouse et al., Nuc. Acids. Res., 1993, 21:2265-2266.

The antibodies of the present invention can also comprise single chain antibodies, which are typically formed by linking the heavy and light chain fragments of the Fv region via an amino acid bridge, resulting in a single chain polypeptide. See e.g., U.S. Pat. No. 4,946,778; Bird, Science, 1988, 242:423-426; Huston et al., Proc. Natl. Acad. Sci. USA, 1988, 85:5879-83; and Ward et al., Nature, 1989, 334:544-546).

Antibody fragments useful in the invention include antigen-binding fragments (i.e., CD20-binding, CD23-binding, and CD80-binding fragments) comprising a CH2 domain and an ability to mediate antibody effector functions, for example, by binding of Fc receptors (FcR) or by binding complement. Antibody fragments may be generated by known techniques, for example, Fab expression libraries may be constructed (Huse et al., Science, 1989, 246:1275-1281) to allow rapid and easy identification of monoclonal Fab fragments with the desired specificity.

Specific binding of an antibody or antibody fragment to the antigens described herein refers to preferential binding of an antibody to the antigen in a heterogeneous sample comprising multiple different antigens. Substantially lacking binding describes a level of binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

### I.A. Anti-CD20 Antibodies

Non-fucosylated anti-CD20 antibodies are prepared by expressing a nucleic acid encoding an anti-CD20 antibody in a cell that lacks one or more proteins required for fucosylation of oligosaccharide moieties, as described in Example 1. A representative anti-CD20 useful in the invention is rituximab (RITUXAN®). The amino acid and nucleic acid sequences of rituximab and other anti-CD20 antibodies, and methods of obtaining anti-CD20 antibodies useful in the invention, are disclosed in U.S. Pat. No. 5,736,137, which is incorporated herein by reference in its entirety. A transfectoma expressing rituximab was deposited on Nov. 4, 1992, with the American Type Culture Collection (ATCC), currently located at 10801 University Boulevard, Manassas, Va. 20110-2209, under the provision of the Budapest Treaty for the International Recognition of the Deposit of Microorganisms for the Purpose of Patent Procedure (“Budapest Treaty”) (ATCC Accession No. 69119).

Additional representative non-fucosylated anti-CD20 antibodies include antibodies and CD20-binding fragments thereof, which comprise a CH2 domain and an ability to mediate antibody effector functions, that compete with rituximab for binding to CD20 in a binding inhibition assay; anti-CD20 antibodies and fragments thereof, which comprise a CH2 domain and an ability to mediate antibody effector functions, and which bind a same epitope as an epitope bound by rituximab; anti-CD20 antibodies and fragments thereof, which comprise a CH2 domain and an ability to mediate antibody effector functions, comprising the variable regions of the rituximab antibody, or variable regions derived from rituximab variable regions, for example, by introduction of one or more amino acid additions, substitutions, or other mutations; anti-CD20 antibodies and fragments thereof, which comprise a CH2 domain and an ability to mediate antibody effector functions, comprising the complementarity determining regions of rituximab.

### I.B. Anti-CD80 Antibodies

Non-fucosylated anti-CD80 antibodies are prepared by expressing a nucleic acid encoding an anti-CD80 antibody in a cell that lacks one or more proteins required for fucosylation of oligosaccharide moieties, as described in Example 1. A representative anti-CD80 useful in the invention is galiximab, which is alternatively referred to as PRIMATIZED® 16C10, IDEC-114, or a PRIMATIZED® antibody having variable regions produced by the antibody produced by the hybridoma of ATCC Accession No. HB-12119. HB-12119 hybridoma was deposited on May 29, 1996, with the ATCC under the provision of the Budapest Treaty. Galiximab is an immunoglobulin (Ig) G1 lambda monoclonal antibody with human constant regions and primate (cynomologous macaque) variable regions. The amino acid and nucleic acid sequences of galiximab and other anti-CD80 antibodies, and methods of obtaining anti-CD80 antibodies useful in the invention, are disclosed in U.S. Pat. No. 6,113,898, which is hereby incorporated by reference in its entirety. Galiximab has been shown to bind CD80 on malignant B cells, and to block CD80-CD28 interaction without interfering with the interaction between CD80 and CD152 (CTLA-4).

Additional representative anti-CD80 antibodies include antibodies and CD80-binding fragments, which comprise a CH2 domain and an ability to mediate antibody effector functions, that compete with galiximab for binding to human CD80 in a binding inhibition assay. Such binding inhibition assays are well-known to the skilled artisan. Non-fucosylated antibodies of the invention also include anti-CD80 antibodies fragments thereof, which comprise a CH2 domain and an ability to mediate antibody effector functions, that bind to the same CD80 epitope as galiximab. Anti-CD80 antibodies that compete for binding to CD80 with galiximab or that bind to the same epitope as galiximab are disclosed in U.S. Pat. No. 7,153,508, which is hereby incorporated by reference in its entirety. Methods for determining the epitope specificity of antibodies can be determined by competition assays, or by in vitro assays such as a radioimmunoassay (RIA) or enzyme-linked immunoabsorbent assay (ELISA) using antigen fusion proteins.

The present invention further includes those anti-CD80 antibodies and fragments thereof, which comprise a CH2 domain and an ability to mediate antibody effector functions, comprising the variable regions of galiximab, or variable regions derived from galiximab variable regions, for example, by introduction of one or more amino acid additions, substitutions, or other mutations. Additional anti-CD80 antibodies and CD80 antibody fragments useful in the invention include antibodies having residues comprising the antigen binding domain of galiximab, i.e., the complementarity determining regions of galiximab, a CH2 domain, and an ability to mediate antibody effector functions.

### I.C. Anti-CD23 Antibodies

A representative anti-CD23 useful in the invention is lumiliximab, which is alternatively referred to as IDEC-152, or PRIMATIZED® 5E8 antibody. Lumiliximab is a PRIMATIZED® anti-CD23 immunoglobulin (Ig) G1 lambda monoclonal antibody with human constant regions and primate (cynomologous macaque) variable regions. As described herein, non-fucosylated lumiliximab may be used to induce ADCC of target cells to a greater level than that induced by fucosylated lumiliximab.

Additional representative non-fucosylated anti-CD23 antibodies include antibodies and fragments thereof, which comprise a CH2 domain and an ability to mediate antibody effector functions, that compete with lumiliximab for binding to CD23 in a binding inhibition assay; antibodies and antibody fragments which bind a same epitope as an epitope bound by lumiliximab; anti-CD23 antibodies and fragments thereof, which comprise a CH2 domain and an ability to mediate antibody effector functions, comprising the variable regions of the lumiliximab antibody, or variable regions derived from lumiliximab variable regions, for example, by introduction of one or more amino acid additions, substitutions, or other mutations; anti-CD23 antibodies fragments thereof, which comprise a CH2 domain and an ability to mediate antibody effector functions, comprising the complementarity determining regions of lumiliximab. The amino acid and nucleic acid sequence of lumiliximab (IDEC-152) and other anti-CD23 antibodies useful in the invention, including 5E8 and 6G5, and antibodies that compete with 5E8 and 6G5 antibodies, as well as methods for obtaining such antibodies, are disclosed in U.S. Patent Application No. 6,011,138, which is hereby incorporated by reference in its entirety.

### I.D. Antibody Variants

The non-fucosylated anti-CD20, anti-CD23, and anti-CD80 antibodies described herein above, and variants thereof, may be readily prepared using recombinant DNA technology. Nucleic acids encoding rituximab are described in U.S. Pat. No. 5,736,137. Nucleic acids encoding the variable regions of IDEC-152 are described in U.S. Pat. No. 6,011,138. Nucleic acids encoding galiximab are described in U.S. Pat. No. 7,153,508.

Variants of anti-CD20, anti-CD23, and anti-CD80 antibodies, i.e., variants of rituximab, lumiliximab, and galiximab, may be readily prepared to include various changes, substitutions, insertions, and deletions. For example, antibody sequences may be optimized for codon usage in the cell type used for antibody expression. To increase the serum half life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angel et al., 1993, Mol. Immunol. 30:105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Variants of anti-CD20, anti-CD23, and anti-CD80 antibodies may be produced using standard recombinant techniques, including site-directed mutagenesis, or recombination cloning. A diversified repertoire of anti-CD20, anti-CD23, and anti-CD80 antibodies may be prepared via gene arrangement and gene conversion methods in transgenic non-human animals (U.S. Patent Publication No. 2003/0017534), which are then tested for relevant activities using functional assays. Anti-CD20, anti-CD23, and anti-CD80 antibody variants may also be obtained using an affinity maturation protocol for mutating CDRs (Yang et al., 1995, J. Mol. Biol. 254:392-403), chain shuffling (Marks et al., 1992, Biotechnology (NY) 10:779-83), of mutator strains of E. coli (Low et al., 1996, J. Mol. Biol. 260:359-68), DNA shuffling (Patten et al., 1997, Curr. Opin. Biotechnol. 8:724-33), phage display (Thompson et al., 1996, J. Mol. Biol. 256:77-88, and sexual PCR (Crameri et al., 1998, Nature 391:288-291.

The skilled artisan can use such nucleic acids to produce non-fucosylated antibodies using cells deficient in fucose metabolism as described herein. For example, nucleic acid sequences which encode for an anti-CD20, anti-CD23, or anti-CD80 antibody may be cloned from a hybridoma or cell line which expresses the antibody, by for example polymerase chain reaction using suitable primers. Nucleic acids encoding antibody variable regions may also be prepared by annealing overlapping oligonucleotides encoding the variable regions, or otherwise synthesized based upon known antibody variable region sequences. The nucleic acids encoding the variable regions are ligated to or assembled with nucleic acids encoding a human antibody constant region in a suitable expression vector. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561.

Tetravalent antibodies (H₄L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, by use of heterobifunctional cross-linkers Wolff et al., 1993, Cancer Res. 53:2560-5, or by recombinant production to include a dual constant region Stevenson et al., 1989, Anticancer Drug Des. 3:219-30. Antigen-binding fragments of antibodies of the invention may be prepared, for example, by expression of truncated antibody sequences, or by post-translation digestion of full-length antibodies.

### I.E. Production of Non-Fucosylated Antibodies

For production of non-fucosylated anti-CD20, anti-CD23, and anti-CD80 antibodies, nucleic acids encoding such antibodies are expressed in a cell deficient in fucose metabolism, such that the cells have a reduced ability to fucosylate proteins that are expressed from the cells. Specifically, a cell which produces non-fucosylated antibodies of the present invention are cells wherein the activity of an enzyme relating to the synthesis of an intracellular sugar nucleotide, GDP-fucose and/or the activity of an enzyme relating to the modification of a sugar chain in which 1-position of fucose is bound to 6-position of N-acetylglucosamine in the reducing end through an α-bond in the complex N-glycoside-linked sugar chain is decreased or deleted. For example, enzymes relating to the synthesis of GDP-fucose include GMD (GDP-mannose 4,6-dehydratase), Fx (GDP-keto-6-deoxymannose 3,5-epimerase, 4-reductase), GFPP (GDP-beta-L-fucose pyrophosphorylase). Representative techniques for generating such cells include (a) disruption of a gene encoding the enzyme; (b) introduction of a dominant negative mutant of a gene encoding the enzyme; (c) mutation of the enzyme; (d) inhibiting transcription and/or translation of a gene encoding the enzyme; and (e) selecting a cell line resistant to a lectin which recognizes a sugar chain in which 1-position of fucose is bound to 6-position of N-acetylglucosamine in the reducing end through α-bond in the complex N-glycoside-linked sugar chain.

For example, cells useful for production of non-fucosylated antibodies may be deficient in a fucose metabolism gene such as a gene encoding for an enzyme relating to the synthesis pathway of the intracellular sugar nucleotide, GDP-fucose. Such genes include those encoding for enzymes including GDP-mannose 4,6-dehydratase, GDP-keto-6-deoxymannose 3,5-epimerase 4,6-reductase, GDP-beta-L-fucose pyrophosphorylase, fucokinase and the like. Methods for disrupting or mutating such genes or inhibiting their transcription or translation are well known in the art. Additional fucose metabolism genes may encode an enzyme which transfers fucose at the 1 position to the 6-position of N-acetylglucosamine in the reducing end of an N-linked oligosaccharide chain via an α-bond may be disrupted or mutated in cells. Such enzymes include α-1,6-fucosyltransferase, α-L-fucosidase and the like.

Cells deficient in fucose metabolism that may be used to produce non-fucosylated antibodies include animal cells, yeast cells, insect cells and plant cells. Non-limiting examples of animal cells include a CHO cell derived from a Chinese hamster ovary tissue, a rat myeloma cell line such as YB2/3HL.P2.G11.16Ag.20 cell, a mouse myeloma cell such as an NS0 cell or a SP2/0-Ag14 cell, a cell derived from a syrian hamster kidney tissue such as a BHK cell, an antibody producing-hybridoma cell, a human leukemia cell line, an embryonic stem cell, a fertilized egg cell, and the like.

Cells useful for expressing non-fucosylated anti-CD20, anti-CD23, and anti-CD80 antibodies may be derived from cell lines deficient in fucose metabolism which are known in the art, such as Ms704, Ms705, and Ms709 cell lines. These cell lines were created by the targeted disruption of a fucose metabolism gene such as the fucosyltransferase, FUT8 (alpha (1,6) fucosyltransferase) in CHO/DG44 cells using two replacement vectors (see e.g. U.S. Patent Publication No. 20040110704). Other useful cell lines include those with a naturally low enzyme activity for adding fucose to the N-acetylglucosamine that binds to the Fc region of the antibody or does not have the enzyme activity, for example the rat myeloma cell line YB2/0 (ATCC Accession No. CRL 1662).

Cells which produce non-fucosylated antibodies may be selected by demonstrating resistance to a lectin, which recognizes an N-linked glycoside oligosaccharide chain having a fucose at the one position bound to a 6 position of N-acetylglucosamine in the reducing end through an α-bond in the N-glycoside-linked oligosaccharide chain. Such resistant cell lines produce antibodies having N-linked glycoside oligosaccharide chains with reduced amounts of fucose bound to the oligosaccharide chains. For example, a lectin-resistant cell line can be obtained by culturing a cell line in a medium comprising a predetermined concentration of lectin and then by selecting a cell line which acquires lectin resistance such that its survival rate is increased at least 2 times, 3 times, or more than 5 times or more, in comparison to a parent cell line. Also, such cell lines may be obtained by culturing a cell line in a medium comprising lectin and then by selecting a cell line which can be cultured at a certain survival rate, e.g., demonstrating a 80% or 90% survival rate, at a lectin concentration of at least 2 times, 5 times, more than 10 times, or more than 20 times or more, than the survival rate of the parent cell line. Any lectin which recognizes a N-linked glycoside linked oligosaccharide chain having fucose moieties may be used. Examples of such lectins include Lens culinaris lectin LCA (lentil agglutinin derived from Lens culinaris), a pea lectin PSA (pea lectin derived from Pisum sativum), a broad bean lectin VFA (agglutinin derived from Vicia faba), and an Aleuria aurantia lectin AAL (lectin derived from Aleuria aurantia) and the like.

An example of a lectin-resistant cell line which may be used to produce non-fucosylated antibodies is the lectin-resistant Chinese Hamster Ovary (CHO), Lec 13. This mutant displays a defective fucose metabolism and therefore has a diminished ability to attach fucose to complex carbohydrates. This cell line may be obtained from Albert Einstein College of Medicine of Yeshiva University, Bronx, N.Y. Lec13 is believed to lack the transcript for GDP-D-mannose-4,6 dehydratase. GDP-D-mannose-4,6-dehydratase generates GDP-mannose-4-keto-6-D deoxymannose from GDP-mannose, which is then converted by 3,5-epimerase 4,6-reductase to L-fucose.

Representative methods for production of non-fucosylated antibodies are set forth in Example 1. See also U.S. Patent Publication No. 2004/0093621, U.S. Pat. No. 6,946,292, PCT International Publication No. WO 2006/089232, and European Published Application No. 1176195, each of which is incorporated by reference in its entirety herein.

Glycosylation of antibody Fc regions, or a change in glycosylation, can be analyzed using mass spectrometric methods, which are known in the art. Representative methods include for example, quadrupole ion trap mass spectrometry (Weiskopf et al., Rapid Commun. Mass Spectrom., 1997, 11(14):1493-504), electrospray ionization-ion trap mass spectrometry (Weiskopf et al., Anal Chem., 1998, 70(20):4441-4447), matrix-assisted laser desorption/ionization time-of-flight (MALDI-TOF) mass spectrometry (Bacher et al., J. Mass Spectrom., 2001, 36(2):124-139), and matrix-assisted laser desorption/ionization quadrupole ion trap time-of-flight (MALDI-QIT-TOF) mass spectrometry (Ojima et al., J. Mass Spectrom., 2005, 40(3):380-388).

### I.F. Enhanced Effector Functions of Non-Fucosylated Antibodies

Non-fucosylated antibodies of the invention have enhanced effector functions when compared to a control fucosylated antibody, i.e., an antibody comprising a substantially same amino acid sequence as antibody to be tested, and which is produced in a cell capable of fucosylation of oligosacdharide chains. Accordingly, a control fucosylated antibody may comprise an Fc region and N-glycoside-linked sugar chains bound to the Fc region, wherein among the total N-glycoside-linked sugar chains bound to the Fc region of the antibody, the ratio of a sugar chain in which fucose is not bound to N-acetylglucosamine in the reducing end in the sugar chain is less than 20%, for example, less than 10%, or approximately zero. A control fucosylated antibody may also comprise an antibody preparation, wherein antibodies in the preparation comprise an Fc region and N-glycoside-linked sugar chains bound to the Fc region, and wherein the percentage of antibody molecules in the preparation having one or more fucose moieties bound to N-acetylglucosamine in the reducing end in the sugar chain is less than about 20%, for example, less than 10%, or approximately zero.

The enhanced effector functions of non-fucosylated antibodies of the invention include increased antibody dependent cellular cytotoxicity; increased complement dependent cytotoxicity; increased binding to immune effector cells, such as natural killer cells, macrophages, polymorphonuclear cells, monocytes, and other FcR-expressing cells; increased apoptosis; increased T cell priming, and antibody dependent cellular phagocytosis. The enhancement of effector functions is mediated by improved binding to Fc receptors expressed by immune effector cells, as compared to control fucosylated antibodies. The crosslinking of Fc receptors on antibody-coated cells triggers signaling pathways that elicit cell engulfment, cell killing, and/or apoptosis. Techniques for assaying the above-noted effector functions are well known in the art.

For treatment of autoimmune/allergic indications, relevant effector functions include inhibition of production or activity of IgE, which may be enhanced by use of a non-fucosylated anti-CD23 antibody. Non-fucosylated antibodies may also show enhanced inhibition of regulatory T cell functions, such as secretion of lymphokines including interleukin 9 (IL-9), interleukin 10 (IL-10), and transforming growth factor-beta (TGF-β); inhibition of Th1 help for cell-mediated immunity and inflammation; inhibition of Th2 help for antibody production; and inhibition of CD8+cytotoxic T lymphocytes. Techniques for assaying these antibody effector functions are well known in the art.

The Fc regions of IgG antibodies (i.e., IgG1, IgG2, IgG3, and IgG4) bind to Fc receptors which are constitutively or inducibly expressed on the surface of phagocytes, including FcγR1 (including isoforms FcγR1A and FcγR1B) which is expressed on macrophages, neutorphils, eosinophils, and dendritic cells; FcγRIIA, which is expressed on macrophages, neutrophils, eosinophils, platelets, and Langerhans cells; FcγRIIB2, which is expressed on macrophages, neutrophils, and eosinophils; FcγRIIB1, which is expressed on B cells and mast cells; FcγRIII (including the FcγRIIIA isoform and 158V, 158V/F, and 158F allotypes), which is expressed on natural killer cells, eosinophils, macrophages, neutrophils, basophils, and γδ T cells. Upon binding of Fc regions, Fcγ receptors on natural killer cells are activated to destroy antibody-coated targets. Binding of Fcγ regions on phagocytes are activated and enable engulfment of antibody-coated pathogens or cells. The Fc regions of IgE antibodies bind to FcER1 receptors on mast cells, basophils, and esoinophils, which then release inflammatory mediators. The Fc regions of IgA and IgM antibodies are bound by FcaR1 expressed on macrophages, neutrophils, and eosinophils, and/or Fcα/μR receptors expressed on macrophages and B cells. These activated accessory cells contribute to uptake and induction of killing of antibody-coated cells. The Fc portions of antibodies also bind complement and initiate the complement cascade, which leads to recruitment and activation of phagocytes and/or direct pathogen destruction.

Fc regions of a non-fucosylated antibody, as described herein, bind to FcRs with an affinity that is at least about 2-fold greater than the affinity of an Fc region of a control fucosylated antibody which has one or more oligosaccharide chains comprising fucose, for example, at least about 5-fold greater affinity, including 10-fold, 20-fold, 30-fold, 50-fold, 100-fold, 500-fold or 1000-fold or more greater affinity. Assays that may be used to determine the ability of antibodies to bind to FcRs are known in the art, for example, as described in PCT International Publication No. WO 00/42072, PCT International Publication No. WO 03/035835, and U.S. Pat. No. 6,242,195.

As described herein, binding of non-fucosylated antibodies to cells expressing all three FcγRIII receptor allotypes is enhanced as compared to control non-fucosylated antibodies. The non-fucosylated antibodies of the present invention may be alternatively characterized as binding to cells expressing the 158 homozygous phenylalanine allotype FcγRIII receptor or to cells expressing the heterozygous 158 valine/phenyalanine allotype FcγRIII receptor with an affinity that is similar or greater than binding of a control fucosylated antibody to cells expressing the homozygous valine allotype FcγRIII receptor.

Non-fucosylated antibodies of the invention exhibit enhanced ADCC activity as compared to control fucosylated antibodies. Enhancement of ADCC activity refers to any measurable increase in antibody dependent cell lysis or phagocytosis that occurs when cells are contacted with a non-fucosylated antibody when compared to a level of ADCC activity induced when cells are contacted with a control fucosylated antibody. For example, non-fucosylated antibodies of the invention may result in a level of ADCC activity that is at least about 10% greater than a level of ADCC activity which occurs when contacted with a control fucosylated anti-CD20, anti-CD23, or anti-CD80 antibody, for example, at least about 20% greater, or at least about 30% greater, or at least about 40% greater, or at least about 50% greater, or at least about 60% greater, or at least about 70% greater, or at least about 80% greater, or at least about 90% greater, or at least about 100% greater, or at least about 150% greater, or more. The non-fucosylated antibodies of the invention may also induce a level of ADCC that is at least about 2-fold greater than the ADCC of the same cells induced by a control fucosylated antibody, for example, at least about 5-fold greater affinity, including 10-fold, 20-fold, 30-fold, 50-fold, 100-fold, 500-fold or 1000-fold or more.

The increased ADCC activity of a non-fucosylated antibody of the invention may also be quantified as an increased potency, as measured by a decrease in the EC₅₀ value for the non-fucosylated form of the antibody, as compared to the control fucosylated form. For example, the EC₅₀ ratio of the fucosylated form to the non-fucosylated form for ADCC of CD20+, CD23+, or CD80+cells is at least 3 (i.e., the EC₅₀ of the non-fucosylated form is 3-fold lower than the EC₅₀ of the fucosylated form), or an EC₅₀ ratio that is at least 4, or at least 5, or at least 7, or at least 10, or at least 15, or at least 20, or greater.

The ADCC activity of non-fucosylated and control fucosylated antibodies can be tested in established in vitro assays. For example, a chromium release ADCC assay may be performed as described in Example 2. Donor cells may be obtained by Ficoll Rypaque density centrifugation, followed by lysis of contaminating erythrocytes. Washed PBMCs can be suspended in RPMI supplemented with 10% heat-inactivated fetal calf serum and mixed with chromium labeled cells expressing CD20, CD23, or CD80, at various ratios of effector cells to tumor cells. Non-fucosylated anti-CD30, anti-CD23, or anti-CD80 antibodies are applied at various concentrations. Samples can be assayed for cytolysis by measuring chromium released into the culture supernatant.

An alternative assay that can be used to assess cell killing, including ADCC activity, is a time resolved fluorometry assay. Briefly, cells expressing CD20, CD23, or CD80 cells are loaded with an acetoxymethyl ester of fluorescence enhancing ligand (BATDA), which penetrates cell membranes. Inside the cell, the ester bonds are hydrolized and the compound can no longer pass the cell membrane. Non-fucosylated antibodies or control fucosylated antibodies are added at various concentrations. Following cytolysis, an europeum solution (Perkin Elmer) is added and any free ligand binds the europeum to form a highly fluorescent and stable chelate (EuTDA) that can be read on a microplate reader (Perkin Elmer). The measured signal correlates with the amount of lysed cells.

### II. Therapeutic Applications

The present invention also provides methods of treating a disease, including neoplastic disorders as well as autoimmune/allergic disorders. The disclosed methods comprise administering to a subject in need thereof, such as a mammal, for example, a human, a therapeutically effective amount of a non-fucosylated anti-CD20, anti-CD23, or anti-CD80 antibody, i.e., an antibody comprising an Fc region and complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is at least 20%.

### II.A. Indications

The disclosed non-fucosylated antibodies are useful for inhibiting growth of cancerous cells and cells of a non-neoplastic proliferative disorder, such as hyperplasia, metaplasia, or most particularly, dysplasia (for review of such abnormal growth conditions, see DeVita, Jr. et al., Cancer: Principles and Practice, 6th edition, 2001, Lippincott Williams & Wilkins.

For example, the disclosed non-fucosylated antibodies may be used to treat B cell malignancies, e.g., leukemias and lymphomas, including indolent, aggressive, low-grade, intermediate-grade, or high-grade leukemia or lymphoma. Representative B cell malignancies include Hodgkin's lymphoma, B cell chronic lymphocytic leukemia (B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved: Burkitt's and non-Burkitt's: follicular, predominantly large cell; follicular, predominantly small cleaved cell; and follicular, mixed small cleaved and large cell lymphomas. Subjects having any of the above-identified B cell malignancies include relapsed subjects, or subjects who are refractory to prior therapy.

Additional cancers that may be treated using the disclosed non-fucosylated antibodies include primary and metastatic tumors in breast, colon, rectum, lung, oropharynx, hypopharynx, esophagus, stomach, pancreas, liver, gallbladder, bile ducts, small intestine, urinary tract including kidney, bladder and urothelium, female genital tract, cervix, uterus, ovaries, male genital tract, prostate, seminal vesicles, testes, an endocrine gland, thyroid gland, adrenal gland, pituitary gland, skin, bone, soft tissues, blood vessels, brain, nerves, eyes, meninges. Other relevant solid tumors include those having B cell involvement.

Representative autoimmune/allergic disorders that may be treated using the disclosed non-fucosylated antibodies include atopic dermatitis, eczema, allergic rhinitis, conjuntivitis, Job's syndrome, and asthma. Additional disorders include allergic bronchopulmonary aspergillosis; allergic rhinitis and conjunctivitis autoimmune hemolytic anemia; acanthosis nigricans; allergic contact dermatitis; Addison's disease; atopic dermatitis; alopecia greata; alopecia universalis; amyloidosis; anaphylactoid purpura; anaphylactoid reaction; aplastic anemia; angioedema, hereditary; angioedema, idiopathic; ankylosing spondylitis; arteritis, cranial; arteritis, giant cell; arteritis, takayasu's; arteritis, temporal; asthma; ataxia-telangiectasia; autoimmune oophoritis; autoimmune orchitis; autoimmune polyendocrine failure; behcet's disease; berger's disease; buerger's disease; bronchitis; bullous pemphigus; candidiasis, chronic mucocutaneous; caplan's syndrome; post-myocardial infarction syndrome; post-pericardiotomy syndrome; carditis; celiac sprue; chagas's disease; chediak-higashi syndrome; churg-strauss disease; cogan's syndrome; cold agglutinin disease; crest syndrome; crohn's disease; cryoglobulinemia; cryptogenic fibrosing alveolitis; dermatitis herpetifomis; dermatomyositis; diabetes mellitus; diamond-blackfan syndrome; digeorge syndrome; discoid lupus erythematosus; eosinophilic fasciitis; episcleritis; drythema elevatum diutinum; erythema marginatum; erythema multiforme; erythema nodosum; familial mediterranean fever; felty's syndrome; fibrosis pulmonary; glomerulonephritis, anaphylactoid; glomerulonephritis, autoimmune; glomerulonephritis, post-streptococcal; glomerulonephritis, post-transplantation; glomerulopathy, membranous; goodpasture's syndrome; graft-vs.-host disease; granulocytopenia, immune-mediated; granuloma annulare; granulomatosis, allergic; granulomatous myositis; grave's disease; hashimoto's thyroiditis; hemolytic disease of the newborn; hemochromatosis, idiopathic; henoch-schoenlein purpura; hepatitis, chronic active and chronic progressive; histiocytosis.sub.-x; hypereosinophilic syndrome; idiopathic thrombocytopenic purpura; job's syndrome; juvenile dermatomyositis; juvenile rheumatoid arthritis (juvenile chronic arthritis); kawasaki's disease; keratitis; keratoconjunctivitis sicca; landry-guillain-barre-strohl syndrome; leprosy, lepromatous; loeffler's syndrome; lupus; lyell's syndrome; lyme disease; lymphomatoid granulomatosis; mastocytosis, systemic; mixed connective tissue disease; mononeuritis multiplex; muckle-wells syndrome; mucocutaneous lymph node syndrome; mucocutaneous lymph node syndrome; multicentric reticulohistiocytosis; multiple sclerosis; myasthenia gravis; mycosis fungoides; necrotizing vasculitis, systemic; nephrotic syndrome; overlap syndrome; panniculitis; paroxysmal cold hemoglobinuria; paroxysmal nocturnal hemoglobinuria; pemphigoid; pemphigus; pemphigus erythematosus; pemphigus foliaceus; pemphigus vulgaris; pigeon breeder's disease; pneumonitis, hypersensitivity; polyarteritis nodosa; polymyalgia rheumatic; polymyositis; polyneuritis, idiopathic; portuguese familial polyneuropathies; pre-eclampsia/eclampsia; primary biliary cirrhosis; progressive systemic sclerosis (scleroderma); psoriasis; psoriatic arthritis; pulmonary alveolar proteinosis; pulmonary fibrosis, raynaud's phenomenon/syndrome; reidel's thyroiditis; reiter's syndrome, relapsing polychrondritis; rheumatic fever; rheumatoid arthritis; sarcoidosis; scleritis; sclerosing cholangitis; serum sickness; sezary syndrome; sjogren's syndrome; stevens-johnson syndrome; still's disease; subacute sclerosing panencephalitis; sympathetic ophthalmia; systemic lupus erythematosus; transplant rejection; ulcerative colitis; undifferentiated connective tissue disease; urticaria, chronic; urticaria, cold; uveitis; vitiligo; weber-christian disease; wegener's granulomatosis; wiskott-aldrich syndrome. Autoimmune/allergic disorders also include inflammatory disorders, including chronic inflammation, such as intestinal inflammation, Crohn's disease, ulcerative colitis, Coeliac disease, proctitis, eosinophilia gastroenteritis, or mastocytosis.

### II.B. Dose and Administration

Non-fucosylated antibodies of the invention may be used to treat neoplastic or autoimmune/allergic disorders by administering to a subject in need thereof a therapeutically effective dose. A therapeutically effective dose refers to an amount of a non-fucosylated anti-anti-CD20, anti-CD23, or anti-80 antibody as described herein sufficient to result in amelioration of symptoms of disease or sufficient to demonstrate a therapeutic effect.

For the treatment of neoplastic diseases, therapeutic effects may be measured using clinical outcomes such as reduction in tumor mass and/or the number of nodules related to a lymphoma neoplastic disorder, reduction of abnormally large spleen or liver, reduction or disappearance of metastases, and progression-free survival. Additional indices of therapeutic effect include reduction of the number of CD20+, CD23+, or CD80+cells, induction of ADCC or complement dependent cytotoxicity (CDC), or reduced or slowed growth of CD20+, CD23+, or CD80+neoplastic cells. The therapeutic effect of the disclosed non-fucosylated antibodies may also be assessed as inhibition of regulatory T cell function to thereby produce an anti-cancer effect. For example, inhibition of regulatory T cell function may be observed as reduced secretion of lymphokines such as interleukin 9 (IL-9), interleukin 10 (IL-10), and transforming growth factor-beta (TGF-β); dis-inhibition of Th1 help for cell-mediated immunity and inflammation; dis-inhibition of Th2 help for antibody production; and dis-inhibition of CD8+ cytotoxic T lymphocytes.

For autoimmune or allergic conditions, a therapeutic effect may be assessed as reduced inflammation, inhibition of IgE production, suppression of humoral immune responses (e.g. production of antigen-specific antibodies), suppression of cell-mediated immune responses (e.g. lymphocyte proliferation), reduced cytokine production, down regulation of self-antigen expression, and/or masking of MHC antigens.

When treating an indication identified herein, a therapeutic effect may be observed as a change in any of the above-noted measurements. The change is assessed relative to a control level or sample, for example a level of a therapeutic index observed in a subject prior to administration of a non-fucosylated antibody, or relative to an effect following administration of a control fucosylated antibody. For example, a change in any of the above-noted indices may be a change of at least about two-fold greater or less than a control level, or at least about at least about five-fold greater or less than a control level, or at least about ten-fold greater or less than a control level, at least about twenty-fold greater or less than a control level, at least about fifty-fold greater or less than a control level, or at least about one hundred-fold greater or less than a control level. A change in the above-noted indices may also be observed as a change of at least 20% compared to a control level, such as at least 30%, or at least 40%, or at least 50%, or at least 60%, or at least 70%, or at least 80%, or at least 90%, or at least 100%, or more.

Toxicity and therapeutic efficacy of non-fucosylated antibodies can be determined by standard pharmaceutical procedures in cell cultures or experimental animals. While compositions which exhibit toxic side effects may be used, care should be taken to design a delivery system that targets such antibodies to the site of affected tissue in order to minimize potential damage to uninfected cells and, thereby, reduce side effects.

The data obtained from the cell-based assays and animal studies, usually in rodents, rabbits, dogs, pigs, and/or or primates, can be used in formulating a range of dosage for use in humans. The animal model may also be used to determine the appropriate concentration range and route of administration. Such information may then be used to determine useful doses and routes for administration in humans. Typically, a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine.

The dosage of such antibodies lies preferably within a range of circulating concentrations with little or no toxicity. The dosage may vary within this range depending upon the dosage form employed and the route of administration utilized. For any present non-fucosylated antibody composition used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

For example, doses for rituximab, galiximab, and lumiliximab, which are prepared in a manner that does not specifically remove fucose residues, may include weekly doses ranging from 100 mg/m² to 600 mg/m², such as 4 weekly infusions at a dose of 125 mg/m², 250 mg/m², 375 mg/m², or 500 mg/m². The non-fucosylated antibodies described herein may be administered at similar doses, or at higher or lower doses. Thus, doses of the non-fucosylated antibodies may include doses that are about 90%, or about 80%, or about 70%, or about 60%, or about 50%, or about 40%, or about 30%, or about 20% or about 10%, or less of the above-identified doses for rituximab, galiximab, and lumiliximab antibodies having fucose residues. For example, effective doses may include weekly doses of 10 mg/m², 12.5 mg/m², 25 mg/m², 37.5 mg/m², or 50 mg/m². In particular, non-fucosylated antibodies having enhanced ADCC activity, or other anti-tumor activity, compared to the same antibody, which is prepared in a manner that does not specifically remove fucose residues, may be effective at a reduced dose. Alternatively, doses of the non-fucosylated antibodies may also include doses that are about 110%, or about 120%, or about 130%, or about 140%, or about 150%, or about 160%, or about 170%, or about 180% or about 190%, or more of the above-identified doses for the rituximab, galiximab, and lumiliximab antibodies having fucose residues.

Pharmaceutical compositions for use in accordance with invention may be formulated in a conventional manner using one or more physiologically acceptable carriers or excipients. Thus, the compositions and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, rectal administration, or within/on implants, e.g., matrices such as collagen fibers or protein polymers, via cell bombardment, in osmotic pumps, grafts comprising appropriately transformed cells, etc.

For treatment of CNS malignancies, non-fucosylated antibodies may be administered to directly to the CNS, for example, by intrathecal or intraventricular administration. In addition, a variety of techniques are available for promoting transfer of antibodies across the blood brain barrier including disruption by surgery or injection, drugs which transiently open adhesion contact between CNS vasculature endothelial cells, and compounds which facilitate translocation through such cells.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Pharmaceutical compositions may also include various buffers (e.g., Tris, acetate, phosphate), solubilizers (e.g., TWEEN®, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as TWEEN®-20, TWEEN®-80, NP-40 or TRITON-X®-100. EBP may also be incorporated into particulate preparations of polymeric compounds for controlled delivery to a patient over an extended period of time. A more extensive survey of components in pharmaceutical compositions is found in Remington's Pharmaceutical Sciences, 1990, 18th ed., A. R. Gennaro, ed., Mack Publishing, Easton, Pa.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

For combination therapies, which include administration of a non-fucosylated antibody along with one or more additional therapeutic agents, administration may be performed within any time frame suitable for performance of the intended therapy. For example, a single agent non-fucosylated antibody and any additional agents may be administered substantially simultaneously (i.e., as a single formulation or within minutes or hours) or consecutively in any order. For consecutive administration, the single agents are administered with an intervening period of about 10, 8, 6, 4, or 2 months, or with an intervening period of 4, 3, 2 or 1 week(s), or with an intervening period of about 5, 4, 3, 2 or 1 day(s).

When used in combination with one or more additional therapeutic agents, the non-fucosylated antibody and the one or more additional agents may be administered or otherwise contacted with cells concurrently or sequentially in either order. The disclosed combination therapies may elicit a synergistic therapeutic effect, i.e, an effect greater than the sum of their individual effects. Measurable therapeutic effects are described herein above. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

For additional guidance regarding formulation, dose, administration regimen, and measurable therapeutic outcomes, see Berkow et al., The Merck Manual of Medical Information, 2000, Merck & Co., Inc., Whitehouse Station, N.J.; Ebadi, CRC Desk Reference of Clinical Pharmacology, 1998, CRC Press, Boca Raton, Fla.; Gennaro, Remington: The Science and Practice of Pharmacy, 2000, Lippincott, Williams & Wilkins, Philadelphia, Pa.; Katzung, Basic & Clinical Pharmacology, 2001, Lange Medical Books/McGraw-Hill Medical Pub. Div., New York; Hardman et al., Goodman & Gilman's the Pharmacological Basis of Therapeutics, 2001, The McGraw-Hill Companies, Columbus, Ohio; Speight & Holford, Avery's Drug Treatment: A Guide to the Properties, Choices, Therapeutic Use and Economic Value of Drugs in Disease Management, 1997, Lippincott, Williams, & Wilkins, Philadelphia, Pa.

### II.C. Combination Therapies

The non-fucosylated antibodies of the present invention may be used in combination with other therapeutic agents or other therapies (e.g., surgical excision, radiation, etc.) to thereby elicit an enhanced therapeutic effect and/or to reduce hepatocytotoxicity of some therapeutic agents. When used in combination with one or more additional therapeutic agents, the non-fucosylated antibodies and the one or more additional agents may be administered or otherwise contacted with cells concurrently or sequentially in either order. As noted herein above, the disclosed combination therapies may elicit a synergistic therapeutic effect, i.e., an effect greater than the sum of their individual effects. Measurable therapeutic effects are described herein above. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

Representative agents useful for combination therapy include cytotoxins, radioisotopes, chemotherapeutic agents, immunomodulatory or immunoregulatory agents, anti-angiogenic agents, anti-proliferative agents, pro-apoptotic agents, and cytostatic and cytolytic enzymes (e.g., RNAses). Additional agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. Non-fucosylated antibodies of the invention may also be used in combination with agents that deplete regulatory T cells, or which block, inhibit, or otherwise downregulate regulatory T cell functions.

The term cytotoxin generally refers to an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine, methotrexate, fluorouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Radioisotopes suitable for radiotherapy include but are not limited to α-emitters, β-emitters, and auger electrons. These radioisotopes are typically conjugated to a targeting antibody for delivery to disease cells. For example, radiolabeled antibodies can include a radioisotope such as ¹⁸-fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ^{121m}tellurium, ⁹⁹technetium, ^{122m}tellurium, ^{125m}tellurium, ¹⁶⁵thulium, ¹⁶⁷thulium, ¹⁶⁸thulium, ⁹⁰yttrium, alpha emitters, such as ²¹³bismuth, ²¹³lead, and ²²⁵actinium, and nitride or oxide forms derived there from.

Immunomodulatory or immunoregulatory agents are compositions that elicit an immune response, including humoral immune responses (e.g. production of antigen-specific antibodies) and cell-mediated immune responses (e.g. lymphocyte proliferation). Representative immunomodulatory agents include cytokines, xanthines, interleukins, interferons, and growth factors (e.g., TNF, CSF, GM-CSF and G-CSF), and hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Immunomodulatory agents useful in the invention also include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

Additional drugs useful in the invention include anti-angiogenic agents that inhibit blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3-VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

Non-fucosylated antibodies of the invention may also be used in combination with other anti-cancer therapeutic antibodies and antibody/drug conjugates. Representative antibodies, which may be used in unlabeled/unconjugated form or as an antibody/drug conjugate, include anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-CD33 antibody/drug conjugates, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

Additional agents that may be used in combination with non-fucosylated antibodies of the invention include agents that block interaction of B lymphocyte stimulator (BLyS) with one or more of its receptors, B cell activating factor receptor (BAFF-R), transmembrane activator and calcium modulator and cyclophilin ligand interactor (TACI), or B-cell maturation antibody (BCMA). For example, useful agents include antibodies that specifically bind the BLyS ligand or antibodies that specifically bind one or more of its receptors, including any of the antibody types described herein. Small molecule inhibitors of the interaction of BLyS with one or more of its receptors may also be used.

Non-fucosylated antibodies of the invention may also be used in combination with systemic anti-cancer drugs, such as epithilones (BMS-247550, Epo-906), reformulations of taxanes (ABRAXANE®, XYOTAX-TM), microtubulin inhibitors (MST-997, TTI-237), hsp90 inhibitors (geldanamycin and derivatives thereof, such as 17-allylamino-17-demethoxygeldanamycin, SNX-5422 (Serenex), STA-9090 (Synta Pharmaceuticals), CCT0180159 (The Institute of Cancer Research), inhibitors of DAX-1, inhibitors of mammalian target of rapamycin/mTOR(CCl-779, AP23573 and RAD-001), etc.

In other combination therapies, non-fucosylated antibodies may be administered together with one or more combinations of chemotherapeutic agents, for example, alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chiorambucil, chlornaphazine, cholophosphamide, estramustine, ifosfamide, mechiorethamine, mechiorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2, 2′, 2′-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology of Princeton, N.J.) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperarnicins; capecitabine and combinations of therapeutic agents such as ABVD (adriamycin, bleomycin, vincristine, dacarbazine).

For the treatment of Hodgkin's Disease, non-fucosylated antibodies of the invention may be used in combination with antibodies that bind to antigens expressed on Hodgkin Reed Sternberg (HRS) cells or cells of the infiltrate surrounding HRS cells. Such antigens that are useful for targeting of HRS cells include CD30, CD40, RANK, TRAIL, Notch, LMP, IL-13, CD20, CD52, and CCR4. Other useful agents for combination therapies for treating Hodgkin's Diseae include proteosome inhibitors (e.g., Bortezomib (PS-341; Millennium Pharmaceuticals) and MG-132 (Tokyo, Metroplitan Institute of Medical Science)), histone deacytylase inhibitors (e.g., depsipeptide (FK228; Gloucester Pharmaceuticals) and suberoylanilide hydroxamic acid (SAHA; Aton Pharma)), and small molecules and peptides that may be used to induce apoptosis of HRS cells, including triterpenoids, such as CDD (RTA401, Reata Discovery) and 17-allylamino-17-demethoxy-gledanamycin (see, e.g., Kamal et al., Trends. Mol. Med., 2004, 10, 283-290), N-acetyl-leucinyl-leucynil-norleucynal, N-acetyl-leucinyl leucynil-methional, carbobenzoxyl-leucinyl-leucynil-norvalinal, carbobenzoxyl-leucinyl-leucynil-leucynal, β-lactone, bactacystine, boronic acid peptides, ubiquitin ligase inhibitors, cyclosporin A, and deoxyspergualin.

For the treatment of autoimmune/allergic disorders, non-fucosylated antibodies of the invention may be used in combination with additional immunoregulatory antibodies (e.g. an anti-CD86 or anti-CD40L antibody) or B cell depleting antibodies (e.g., anti-CD19, anti-CD20, anti-CD22, anti-CD23, or anti-CD37 antibodies). Other useful agents include immunosuppressants, for example, substances that suppress cytokine production, downregulate or suppress self-antigen expression, or mask the MHC antigens. Examples of such agents include 2-amino-6-aryl-5-substituted pyrimidines (U.S. Pat. No. 4,665,077), azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-α, -β, and -δ antibodies, anti-tumor necrosis factor-α antibodies, anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (PCT International Publication No. WO 90/08187), streptolanase; TGF-beta; streptodornase; RNA or DNA from the host; FK506; RS-61443; deoxyspergualin; rapamycin; T-cell receptor (U.S. Pat. No. 5,114,721); T-cell receptor fragments (Offner et al., Science, 1991, 251:430-432; PCT International Publication No. WO 90/11294; Laneway, Nature, 1989, 341:482 (1989); PCT International Publication No. WO 91/01133); and T cell receptor antibodies (European Patent No. 340,109) such as T10B9.

Throughout this application, various publications, patents and published patent applications are referred to by an identifying citation. The disclosures of these publications, patents and published patent specifications referenced in this application are hereby incorporated by reference into the present disclosure to more fully describe the state of the art to which this invention pertains.

The following examples have been included to illustrate modes of the invention. Certain aspects of the following examples are described in terms of techniques and procedures found or contemplated by co-inventors to work well in the practice of the invention. In light of disclosure and the general level of skill in the art, those of skill will appreciate that the following examples are intended to be exemplary only and that numerous changes, modifications, and alterations may be employed without departing from the scope of the invention.

### EXAMPLES

### Example 1

### Preparation of Non-Fucosylated Antibodies

A vector encoding an antibody of interest is transfected into cell lines including a control cell line, i.e. a dihydrofolate reductase-deficient CHO cell line, CHO/DG44, and cells lines known to produce glycoproteins having reduced amounts of fucose moieties in the bound N glycoside-linked oligosaccharides. Representative cell lines include a variant CHO cell line deficient in endogenous GDP-mannose 4, 6 dehydratase (GMD) (Lec13); a rat hybridoma cell line (YB2/0) (available from the American Type Culture Collection of Manassas, Va. (CRL-1662)), and a FUT8 knockout CHO cell line (e.g., Ms704)Cell lines are maintained in Iscove's Modified Dulbecco's Medium (IMDM) medium (Invitrogen) containing 10% (v/v) dialyzed fetal bovine serum (dFBS), 0.1 mM hypoxanthine and 16 μM thymidine. YB2/0 is cultured in GIBCO™ RPMI Medium 1640) containing 10% (v/v fetal bovine serum) (FBS).

Twenty micrograms of the N5LG1 expression vector are transfected into each of the cell lines. The transfected cells are selected in media that requires the activity of DHFR for nucleotide synthesis and cell growth. Exposure of the cells to several rounds of gradually increased concentrations (e.g. from 0 to 500 nM) of the DHFR enzyme inhibitor, methotrexate (MTX), promotes amplification of the DHFR and the co-transfected target gene. MTX treatment enhances anti-CD23 antibody production following an increased gene copy number, which can be up to several hundred in selected cells.

Single cell cloning by limiting dilution is performed to establish high producers after the selected MTX-resistant cells are adapted to serum-free medium containing appropriate concentrations of MTX. EX-CELL™ 302 (JRH Biosciences) supplemented with 6 mM L-gluthamine and HYBRIDOMA-SFM™ (Invitrogen) are employed as the basal serum-free medium for the CHO cells and YB2/0 cells respectively. Lec13 and YB2/0 clones are selected in a medium, which additionally contains 0.5 mg/mL Lens culinaris agglutinin (LCA, Vector Laboratories). LCA recognizes the α-1,6 fucosylated core structure of N-linked oligosaccharides and shows toxicity to cells expressing the recognized oligosaccharide.

After the transfectants are subjected to MTX gene amplification, serum-free medium adaptation and single cell cloning as described, the antibody concentration in the culture supernatant for each of the transfected cells lines is measured by enzyme-linked immunosorbent assay (ELISA). The transfectants result in the generation of clones with relatively high specific production rates of approximately 10-30 pg/cell/day.

Transformed clones are suspended in the serum free media as described above at a density of 1.0×10⁵ cells/ml and dispensed in 50 ml aliquots into 182 cm² flasks. The cells are cultured at 37° C. for 7 days in a 5% CO₂ incubator and the culture supernatant is recovered at confluence. The anti-CD20, anti-CD23, or anti-CD80 antibody is purified from the culture supernatant using a Prosep_A (Millipore) column in accordance with the manufacturer's instructions. About 3 pg of the obtained anti-CD20, anti-CD23, or anti-CD80 antibody is subjected to electrophoresis to examine its molecular weight and purification degree. Oligosaccharides of the anti-CD20, anti-CD23, or anti-CD80 antibody, purified as described above are analyzed. The oligosaccharides are cleaved from proteins by subjecting the antibodies to hydrazinolysis. After removing hydrazine by evaporation under a reduced pressure, N-acetylation is carried out by adding an aqueous ammonium acetate solution and acetic anhydride. After free drying, fluorescence labeling by 2-aminopyridine is performed. A fluorescence-labeled oligosaccharide is separated from excess reagents using SUPERDEX® peptide HR 10/30 column (Pharmacia). The fluorescence-labeled oligosaccharides are dried by centrifugation. The fluorescence-labeled oligosaccharides are subjected to reverse phase HPLC analysis using a CLC-ODS column.

Elution patterns are obtained by reverse phase HPLC analysis for each fraction of fluorescence-labeled oligosaccharides. Differences in the amount of α-1,6 fucose bound oligosaccharides are observed between antibody compositions derived from control and test cells. For control cells, the amount of α-1,6 fucose bound sugar chains is greater than that in the putative non-fucosylated test cells. The variation in the amount of fucose negative products in the test cells may depend on the type of host cells employed.

### Example 2

### Cytotoxic Activity (ADCC Activity) of Non-Fucosylated Antibodies on B Cell Lymphoma Cells

Non-fucosylated lumiliximab and rituximab antibodies were prepared essentially as described in Example 1. The ability of the non-fucosylated antibodies to induce ADCC of CD20+, CD23+SKW B cell lymphoma cells was tested in a cytotoxicity chromium release assay.

Human effector cells were prepared from whole blood from three donors heterozygous (V/F) for the FcγRIIIa receptor at position 158. Briefly, human peripheral blood mononuclear cells were purified from heparinized whole blood by standard Ficoll-paque separation. The cells were resuspended in GIBCO™ RPMI1640 media containing 10% FBS and 200 U/ml of human IL-2 and incubated overnight at 37° C. The following day, the cells were collected and washed once in culture media and resuspended at 1×10⁷ cells/ml.

Target cells were incubated with 100 μCi ⁵¹Cr for 1 hour at 37° C. The target cells were washed once to remove the unincorporated ⁵¹Cr, and plated at a volume of 1×10⁴ cells/well. Target cells were incubated with 50 μl of effector cells and 50 μl of antibody. A target to effector ratio of 1:50 was used throughout the experiments. Five test samples were used. These included (a) target cells in medium; (b) target cells in the presence of 1% TRITON-X®-100; (c) the anti-CD23 antibody lumiliximab; (d) CE9.1, a macaque/human chimeric IgG1 monoclonal antibody (mAb) directed against the human T-lymphocyte receptor, CD4; and (e) the anti-CD20 antibody rituximab (RITUXAN®).

Following a four hour incubation at 37° C., the supernatants were collected and counted on a gamma Counter (Isodata Gamma Counter, Packard Instruments). The counts per minute were plotted as a function of antibody concentration and cell cytotxicity curves from each of the four donors were analyzed using varying concentrations of fucosylated and non-fucosylated antibodies. The percentage lysis of target cells was calculated as follows:

$$\% Lysis = \frac{sample Release ( CPM ) - spontaneous release ( CPM )}{maximum release ( CPM ) - spontaneous release CPM )} \times 100 \%$$

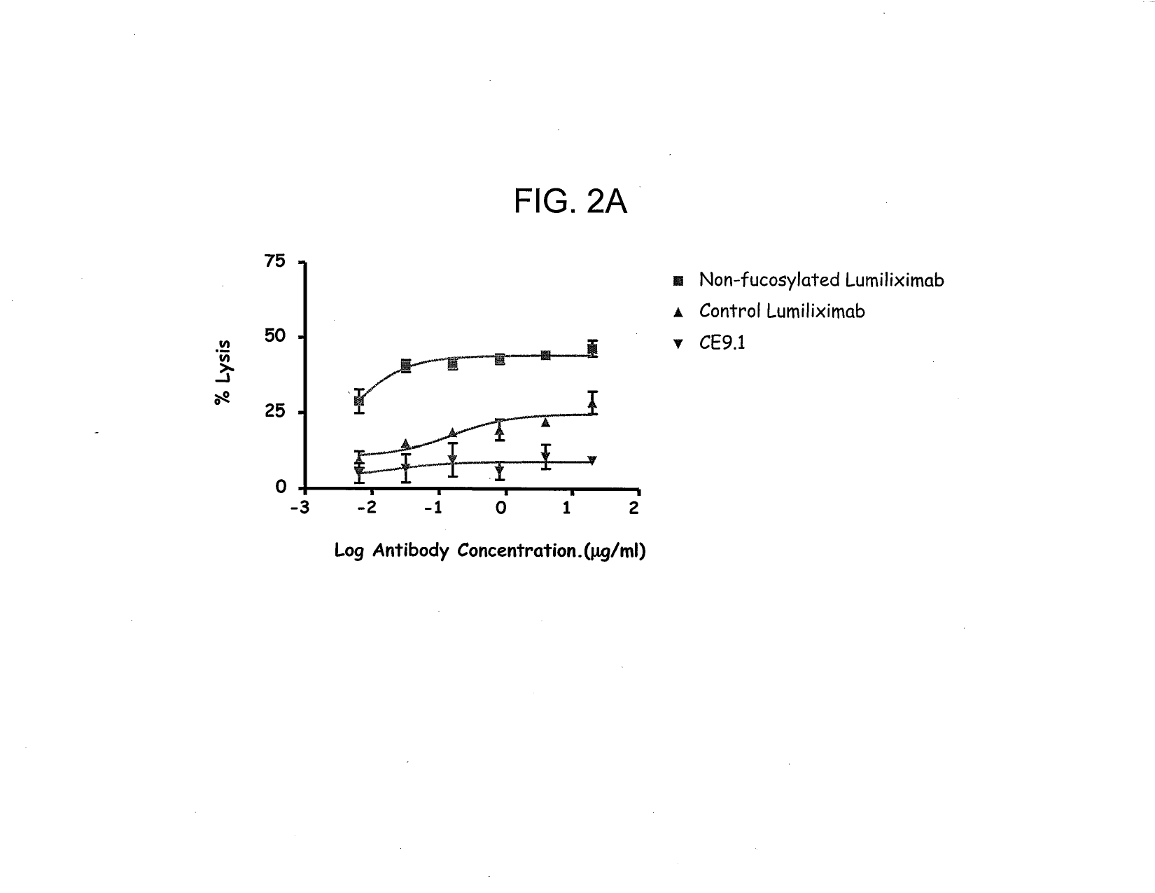

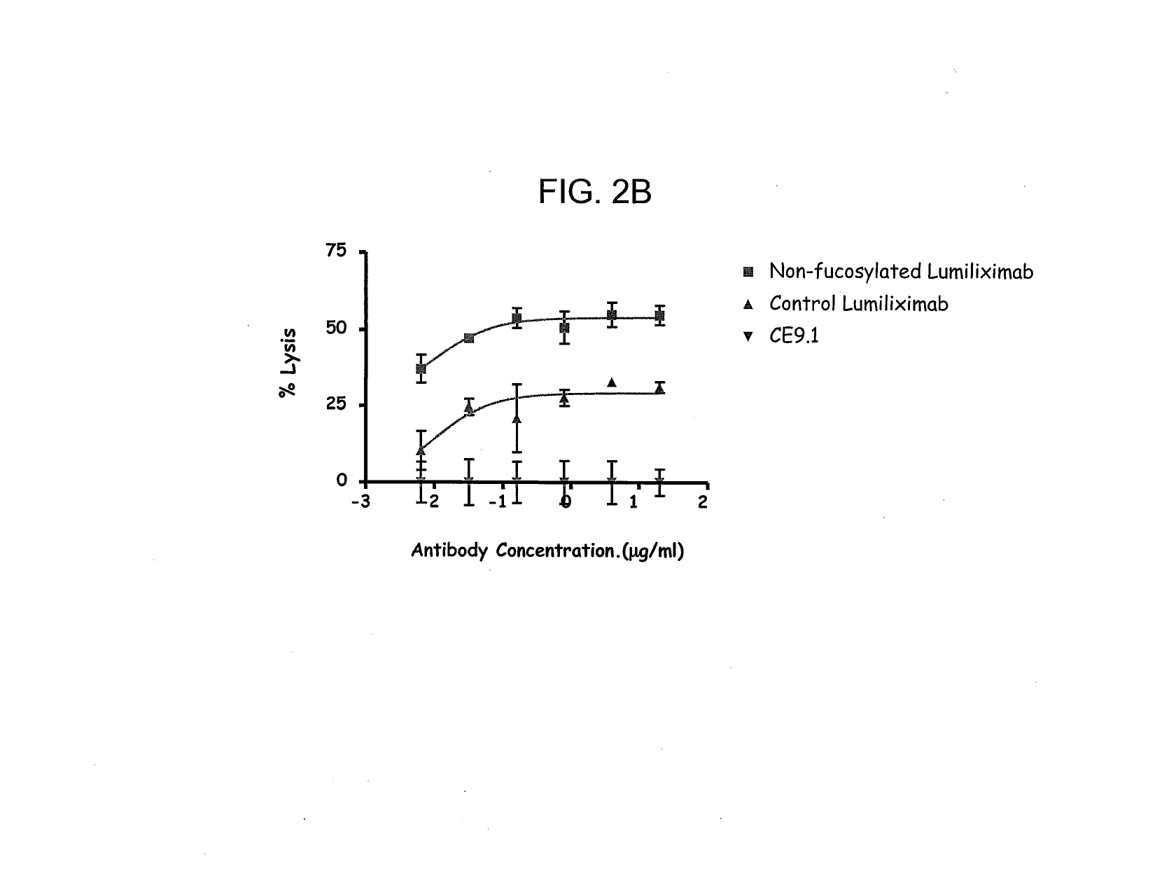

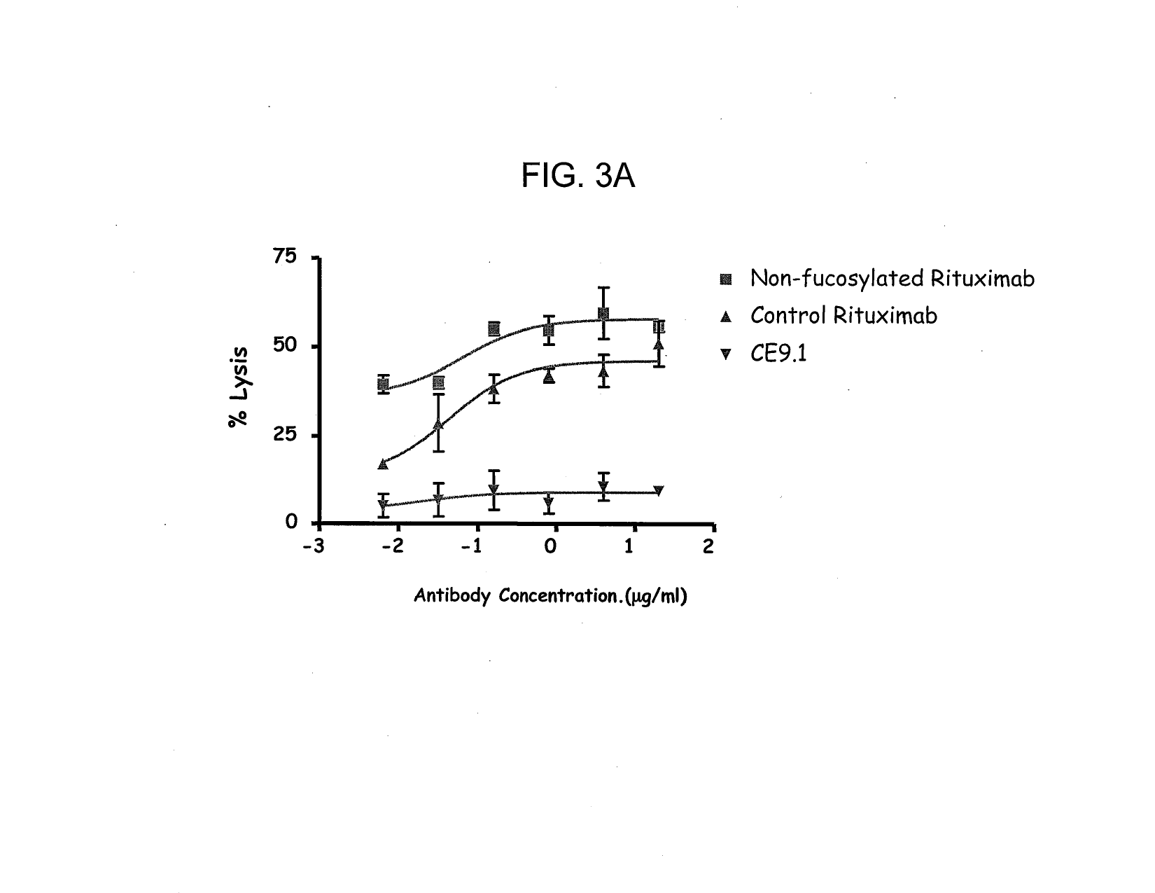

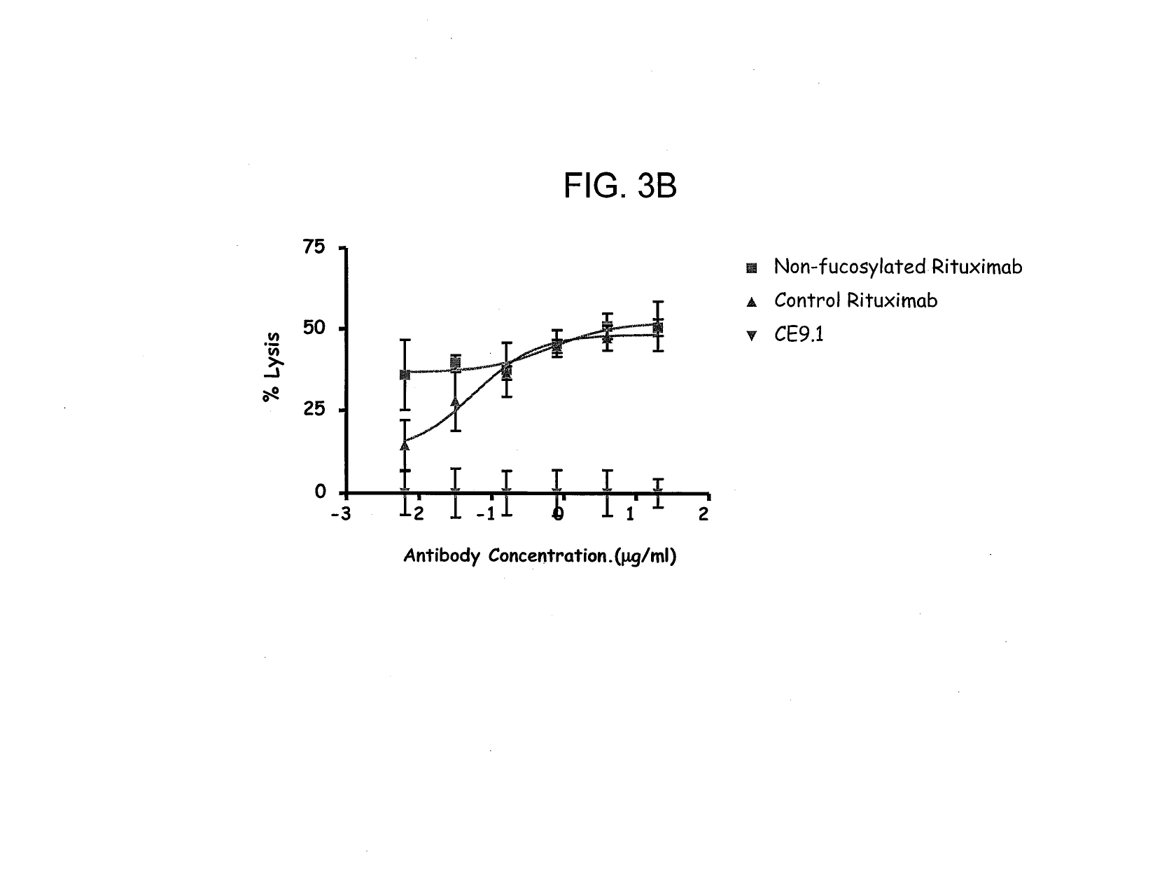

Non-fucosylated lumiliximab antibody exhibited approximately 1-3 times enhanced ADCC as compared to the control lumiliximab antibody when used for lysis of CD20+, CD23+ SKW cells by peripheral blood mononuclear cells from donors 127 and 128. See FIGS. 2A-2B. Non-fucosylated rituximab also showed some increase in ADCC activity. See FIGS. 3A-3B. Table 1 shows the ratio of percentage lysis of CD20+, CD23+SKW tumor cells induced by non-fucosylated antibodies over the percentage lysis induced by fucosylated antibodies.

TABLE 1

| | | Percentage lysis at 1 nM | |
|---|---|---|---|
| PMBC | | Non-fucosylated | Fucosylated |
| Donor | Antibody | Antibody | Antibody |
| 128 | Lumiliximab | 41.1 | 18.6 |
| 128 | Rituximab | 54.9 | 38.4 |
| 127 | Lumiliximab | 53.8 | 21.0 |
| 127 | Rituximab | 37.4 | 36.3 |
| 126 | Lumiliximab | 13.3 | 10.0 |
| 126 | Rituximab | 13.3 | 14.2 |

### Example 3

### Antibody Dependent Cellular Cytoxicity (ADCC) Activity of Non-fucosylated Antibodies on Non-Hodgkin's B cell Lymphoma Cells

Non-fucosylated galiximab (anti-CD80 antibody) was prepared essentially as described in Example 1. The ability of the non-fucosylated antibody to induce ADCC of the CD80+Non-Hodgkin's B-cell lymphoma (B-NHL) Raji cell line was tested by a modified version of the chromium release assay discussed in Example 2.

Isolated and purified Natural Killer (NK) cells were used as human effector cells. Briefly, NK cells were negatively selected from the PBMC population by indirect magnetic labeling of all non-NK cells (T cells, B cells, stem cells, dendritic cells, monocytes, granulocytes, and erythroid cells) using the NK Cell Isolation Kit (Meltenyi Biotec of Aubern, Calif.). The purity of the NK cell population was determined by staining with anti-CD56, anti-CD3 and anti-CD19 antibodies conjugated to phycoerythrin (PE) and subsequent analysis using a FACS Calibur flow cytomter and flow cytometry analysis software. Cells isolated by this procedure were 97% positive for expression of CD56, as opposed to only 8% positive for expression of CD56 in the total PBMC population.

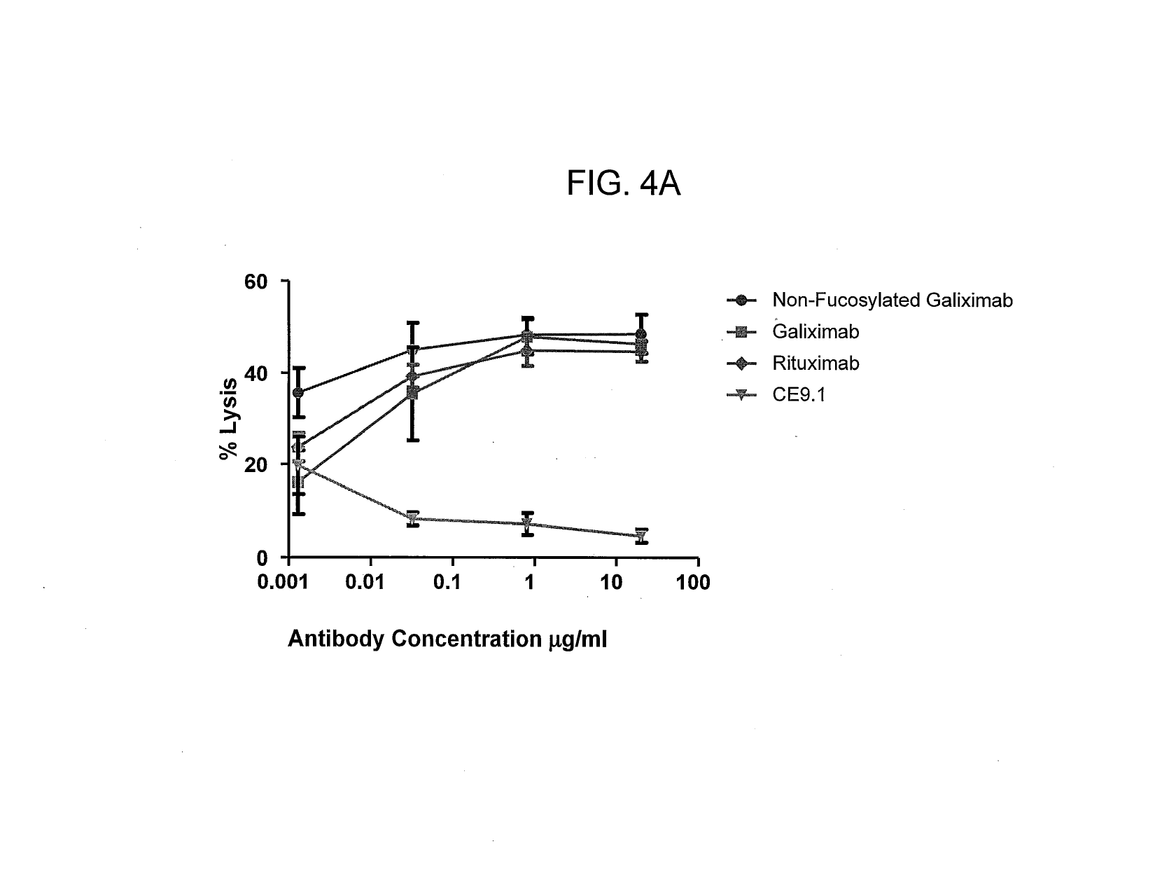

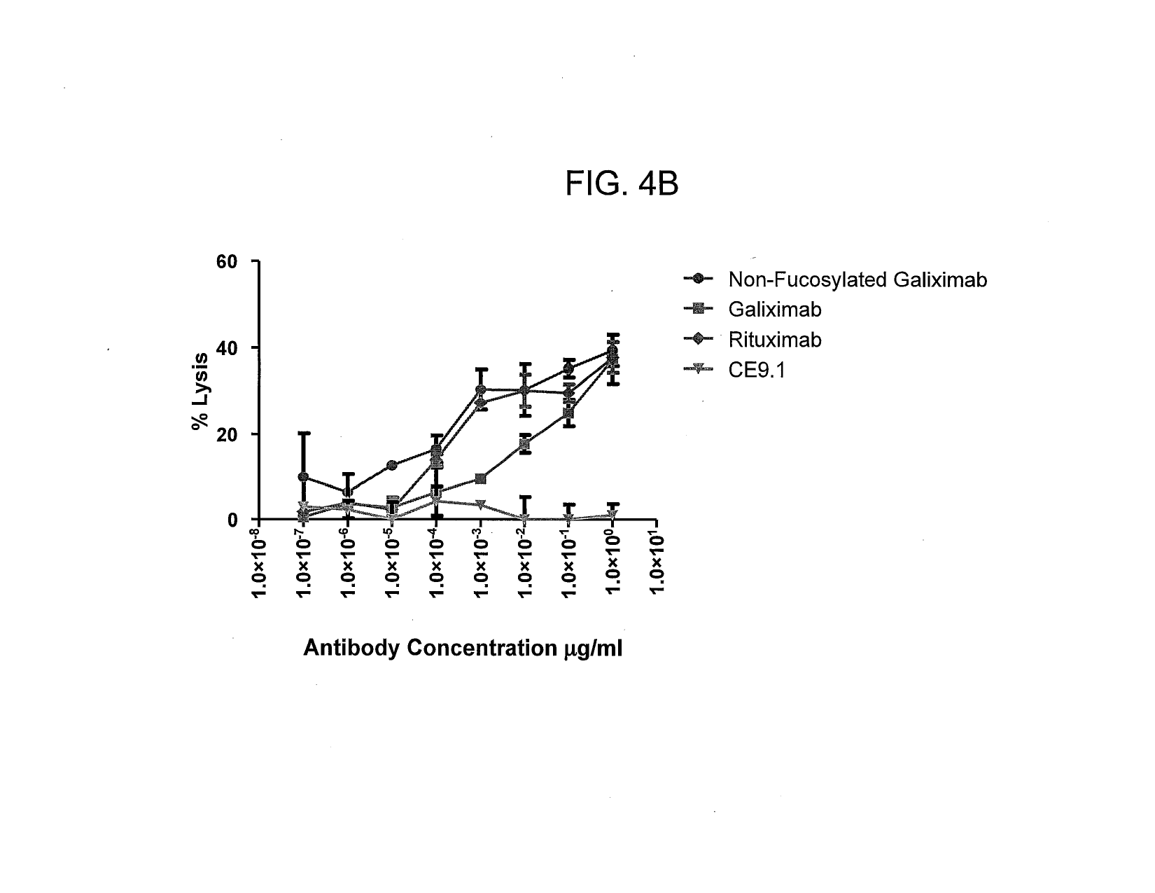

The isolated NK cells were cultured overnight in the presence of 20 U/ml of IL-2 for use as ADCC effector cells. The ability of non-fucosylated galiximab to induce ADCC was assessed by a cytotoxicity chromium release assay as described in Example 2, except that the effector to target ratio was 10:1. Non-fucosylated galiximab, fucosylated galiximab, rituximab(positive control) and anti-CE9.1 (negative control) were added at concentrations of 20, 0.8, 0.032, and 0.0013 μg/ml. Non-fucosylated galiximab exhibited 20% higher ADCC at 0.0013 μg/ml and 10% higher ADCC at 0.032 μg/ml than fucosylated galiximab or rituximab. (FIG. 4A). Additionally, at very low antibody concentrations, non-fucosylated galiximab exhibited at least 10 fold greater potency over unmodified galiximab (FIG. 4B).

### Example 4

### Cytotoxic Activity (ADCC Activity) of Non-Fucosylated Antibodies on Chronic Lymphocytic Leukemia Cells

The ability of the non-fucosylated lumiliximab and rituximab antibodies to induce ADCC of CD20+, CD23+CLL cells isolated from CLL patients was tested by a modified version of the chromium release assay discussed in Example 2.

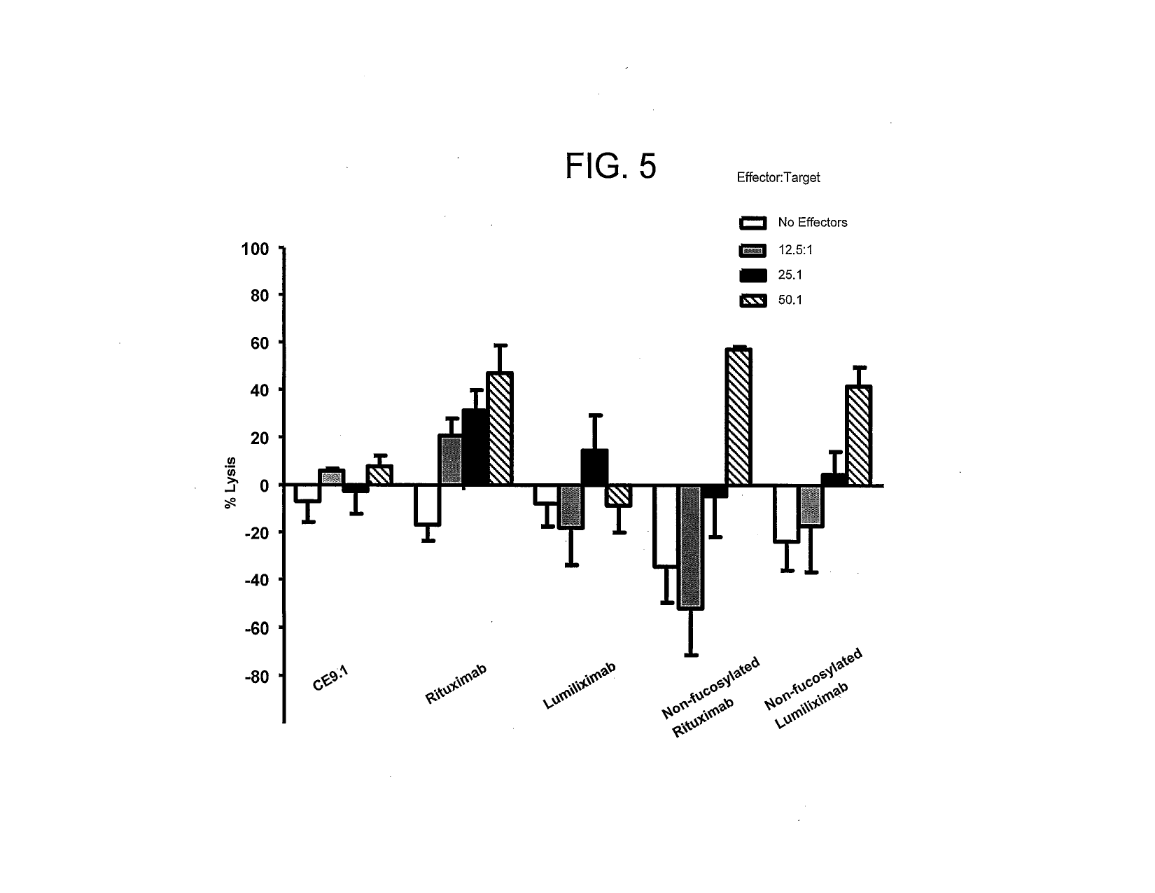

B-CLL cells were isolated from donors 174 and 234, while PMBC effector cells were isolated from donors 210 and 123. Five test samples were used. These included (a) CE9.1, a macaque/human chimeric IgG1 monoclonal antibody (mAb) directed against the human T-lymphocyte receptor, CD4; (b) the anti-CD20 antibody rituximab (RITUXAN®); (c) the anti-CD23 antibody lumiliximab; (d) non-fucosylated rituximab; and (e) non-fucosylated lumiliximab. The same concentration of antibody was used in all tests (1 nM), but the ratio of effector to target cells was varied for each sample. The five test samples were assessed for ADCC of B-CLL cells at effector to target ratios of 0, 12.5:1, 25:1 and 50:1. Similar to the results obtained with the CD20+CD23+SKW lymphoma cells (Example 2), non-fucosylated rituximab exhibited some increase in cell lysis over the control rituximab antibody. See FIG. 5 and Table 2. Likewise, the non-fucosylated lumiliximab exhibited approximately 1-3 times enhanced ADCC as compared to the control lumiliximab antibody. See FIG. 5 and Table 2. Table 2 shows the ratio of percentage lysis of CD20+, CD23+CLL patient B cells induced by non-fucosylated antibodies over the percentage lysis induced by fucosylated antibodies.

TABLE 2

| CLL | | | Percentage lysis at 1 nM | |
|---|---|---|---|---|
| Patient | PMBC | | Non-fucosylated | Fucosylated |
| Donor | Donor | Antibody | Antibody | Antibody |
| 174 | 210 | Lumiliximab | 41.6 | 14.6 |
| 174 | 210 | Rituximab | 56.9 | 46.9 |
| 234 | 123 | Lumiliximab | 52.1 | 25.7 |
| 234 | 123 | Rituximab | 65.3 | 57.1 |

1. An antibody comprising an Fc region and complex N-glycoside-linked sugar chains bound to the Fc region, wherein at least 20% of the total complex N-glycoside-linked sugar chains bound to the Fc region are sugar chains in which fucose is not bound to N-acetylglucosamine in the reducing end in the sugar chain wherein the antibody is selected from an anti-CD23 antibody and an anti-CD80 antibody.

2. The antibody of claim 1, wherein the sugar chain to which fucose is not bound is a high mannose type oligosaccharide, a complex type oligosaccharide, or a hybrid type oligosaccharide.

3. The antibody of claim 2, wherein the sugar chain to which fucose is not bound is a complex N-glycoside-linked sugar chain in which 1-position of fucose is not bound to the 6-position of N-acetylglucosamine in the reducing end through the γ-bond.

4. The antibody of claim 1, wherein the Fc region is a human IgG Fe region.

5. The antibody of claim 4, wherein the human IgG Fc region is a human IgG1, IgG2, IgG3, or IgG4 Fc region.

6. The antibody of claim 4, which binds FcγR receptors with a greater affinity than a control fucosylated antibody.

7. (canceled)

8. The antibody of claim 1, which induces a level of antibody dependent cellular cytotoxicity (ADCC) of cells expressing the target antigen greater than a level of ADCC induced by a corresponding control fucosylated antibody.

9. The antibody of claim 1, wherein the antibody is a chimeric, humanized, or human antibody.

10. The antibody of claim 1, wherein the antibody is an anti-CD23 antibody selected from the group consisting of an anti-CD23 antibody which binds an epitope on human CD23 also bound by lumiliximab, an anti-CD23 antibody which competes for binding to human CD23 with lumiliximab, an anti-CD23 antibody which comprises variable regions derived from variable regions of the antibody lumiliximab, an anti-CD23 antibody which comprises variable regions of lumiliximab, an anti-CD23 antibody which comprises complementarity determining regions (CDRs) of lumiliximab an anti-CD23 antibody which comprises variable regions and constant regions of lumiliximab.

11-15. (canceled)

16. A cell expressing the antibody of claim 1.

17. A method of treating a neoplastic disorder comprising administering to a subject the antibody of claim 1.

18. The method of claim 17, further comprising administering to the subject one or more additional therapeutic agents, wherein in the antibody and the one or more additional therapeutic agents are administered concurrently or consecutively in either order.

19. A method of treating an autoimmune or allergic disorder comprising administering the antibody of claim 1.

20. The method of claim 19, further comprising administering to the subject one or more additional therapeutic agents, wherein in the antibody and the one or more additional therapeutic agents are administered concurrently or consecutively in either order.

21-28. (canceled)

29. The anti-CD80 antibody of claim 1, wherein the antibody is an anti-CD80 antibody selected from the group consisting of an anti-CD80 antibody which binds an epitope on human CD80 also bound by the antibody produced by the hybridoma deposited as ATCC Accession No. HB-12119, an anti-CD80 antibody which competes for binding to human CD80 with the antibody produced by the hybridoma deposited as ATCC Accession No. HB-12119, an anti-CD80 antibody which comprises variable regions derived from variable regions of the antibody produced by the hybridoma deposited as ATCC Accession No. HB-12119, an anti-CD80 antibody which comprises variable regions of the antibody produced by the hybridoma deposited as ATCC Accession No. HB-12119, an anti-CD80 antibody which comprises complementarity determining regions (CDRs) of the antibody produced by the hybridoma deposited as ATCC Accession No. HB-12119 and an anti-CD80 antibody which comprises variable regions and constant regions of the antibody produced by the hybridoma deposited as ATCC Accession No. HB-12119.

30-39. (canceled)
